(19) United States
(12) Patent Application Publication
(10) Pub. No.: US 2005/0260225 A1
(43) Pub. Date: Nov. 24, 2005

(54) Intranasal recombinant Salmonella vaccine encoding heterologous polysaccharide antigens

(76) Inventors: Joanna B. Goldberg, Charlottesville, VA (US); Antonio DiGiandomenico, Charlottesville, VA (US); Jayasimha Rao, Charlottesville, VA (US)

Correspondence Address: Robert G. Rosenthal, Esq., Suite 200, 5856 Faringdon Place, Raleigh, NC, 27609, US

(21) Appl. No.: 11/129,613

(22) Filed: May 13, 2005

Related U.S. Application Data
(60) Provisional application No. 60/572,044, filed on May 18, 2004.
(60) Provisional application No. 60/634,700, filed on Dec. 9, 2004.

(51) Int. Cl. A61K 39/112

(52) U.S. Cl. 424/200.1; 424/258.1

(57) ABSTRACT

The invention relates of administration of an attenuated Salmonella strain expressing a lipopolysaccharide O antigen from a suitable pathogen, in particular Pseudomonas aeruginosa, and the use of the same as a vaccine to promote sterile immunity to the pathogen, e.g., P. aeruginosa, via intranasal vaccination. In one embodiment, the present invention is directed to a unique intranasal route of immunization for the delivery of relevant heterologous polysaccharide antigens via a live, attenuated Salmonella strain.

### BACKGROUND OF THE INVENTION

1. Introduction

This invention relates to immunization by delivery of relevant heterologous polysaccharide antigens via a live, attenuated Salmonella strain. More particularly, this invention relates to an attenuated Salmonella strain expressing a lipopolysaccharide O antigen from a Gram-negative pathogen and the use of the same as a vaccine by intranasal administration. In a preferred embodiment of the invention, this invention relates to an attenuated Salmonella strain expressing a lipopolysaccharide O antigen from Pseudomonas aeruginosa and the use of the same as a vaccine to promote sterile immunity to P. aeruginosa pneumonia via intranasal vaccination.

2. Description of the Prior Art

Vaccines are materials used to protect against diseases caused by pathogens. These pathogens are microbial organisms, such as bacteria and viruses, which affect animals, including humans. Vaccines are intended to produce an immune response in the recipient consisting of at least one antibody-mediated or T cell-mediated immune response, thereby preventing future infection by a pathogen, or fighting a current pathogenic infection. In particular, vaccines against facultative intracellular pathogens, those growing inside the cells of the infected host, need to induce a strong and appropriate cell-mediated immune response. In contrast, vaccines against obligate extracellular pathogens need to induce an appropriate antibody-mediated immune response. Often, regardless of the pathogen, an appropriate combined antibody- and cellular-mediated immune response leads to sufficient protection or relief from infection. In order to achieve this protection or relief from infection, vaccines may express one or more homologous antigens, heterologous antigens, or a combination of both.

Vaccines are primarily derived from a pathogen by producing and administering either: a) an attenuated or a virulent version of the pathogen; b) the killed pathogen; c) extracted protective antigens or antigen mixtures of the pathogen (homologous antigens); or d) a microorganism expressing one or more protective antigens encoded by cloned genes originating in a microbial pathogen different from the vaccine strain (heterologous antigens).

A microorganism expressing a protective antigen originating from a microbial pathogen different from the vaccine strain (d above) is one of the preferred methods used to form vaccines. Bacterial polysaccharides are generally excellent targets for vaccine development as they are surface exposed and often the most protective antigens expressed on a pathogen. However, purified polysaccharides are usually poor immunogens because, unlike proteins, most polysaccharides are not recognized by T lymphocytes. These antigens generally do not elicit an adequate immune response or immunological memory, particularly in young children. Linking polysaccharides to protein carriers can enhance their immunogenicity and induce significant antibody production due to the recruitment of T-cell help. For example, the vaccine to Streptococcus pneumoniae (Pneumovax) contains 23 capsular polysaccharides; and the antibody response to this vaccine is immunologically poor or inconsistent in young children whose immune systems are not fully developed. Because of this, a 7 valent-capsular polysaccharide-protein conjugate (Prevnar) is more effective for young children. Similarly, the polysaccharide-protein-conjugate, Haemophilus influenzae type B vaccine, has had good success in young children.

Live vaccines based on attenuated bacteria have been shown to elicit immune responses of greater magnitude and of longer duration than other types of vaccine constructs. These replicating vaccines have been suggested to confer protection because the duration of the infection resembles the early stages of a natural infection. Salmonella strains have an advantage since they are known to stimulate humoral, cell-mediated, and secretrory immune responses. See Chatfield et al, 1994. Progress in the development of multivalent oral Vaccines based on live attenuated Salmonella. In Modern Vacccinology. E. Kurstak, editor. New York, N.Y.: Plenum Medical Book Company. 55-86, incorporated herein by reference for its discussion of methods used for vaccine formation using attenuated Salmonella. The introduction of specific mutations into Salmonella genomes can render these bacteria attenuated while preserving their invasiveness, immunogenicity, and ability to induce both antibody- and cell-mediated immune responses following administration resulting in organisms suitable for use as vaccine candidates.

A number of attenuated strains of S. typhimurium have been developed as vaccine candidates and used in animal studies. For example, strains of S. typhimurium have been mutated in genes for the synthesis of lipopolysacchrides, amino acids, regulators, or virulence factors. It has been observed that attenuated strains of Salmonella that are similar in their degree of attenuation may vary in the type of immune response that is generated. One of the most studied attenuated S. typhimurium strains is SL3261, which is an aroA mutant. The aroA gene encodes 3-enolpyruvyl-shikimate-5-phosphate synthetase, an enzyme required for the synthesis of aromatic amino acids and growth.

As disclosed by Sirard et al. 1999. Live attenuated Salmonella: a paradigm of mucosal vaccines. Immunol rev 171:5-26, incorporated herein by reference, Salmonella can express heterologous antigens,.including both proteins and polysaccharides, from other pathogens, and elicit strong local and systemic responses after administration at a mucosal surface. These vaccines have been shown to be protective against the carrier organisms as well as the pathogen from which the heterologous antigen originated.

The reason Salmonella is such an effective vaccine carrier lies in its ability to home to cells within the mucosal-associated lymphoid tissue, which is selectively divided into inductive and effector sites and also comprises what has been termed as the common mucosal immune system. Thus, presentation of an antigen at one mucosal site can stimulate immunity at a distant site. For example, oral immunization with S. typhi Ty21a can elicit an immune response in serum, as well as in the intestinal and respiratory tracts of humans. Similar findings have been reported for S. typhimurium in a mouse model system. While the classic mode of delivery of these strains has been by the oral route, more recently there have been reports of intranasal administration of these vaccines. See for example, Pasetti et al, A comparison of immunogenicity and in vivo distribution of Salmonella enterica serovar Typhi and Typhimurium live vector vaccines delivered by mucosal routes in the murine model. Vaccine 18:3208-3213; Corthesy-Theulaz et al. 1998. Mice are protected from Helicbacter pylori infection by nasal immunization with attenuated Salmonella typhimurium phoPc expressing urease A and B subunits. Infect Immun 66:581-586; Pickett et al 2000. In vivo characterization of the murine intranasal model for assessing the immunogenicity of attenuated Salmonella enterica serovar Typhi strains as live mucosal vaccines and as live vectors. Infect Immun 68:205-213 and; Mielcarek et al 2001. Nasal vaccination using live bacterial vectors. Adv Drug Deliv Rev 51:55-69, each incorporated herein by reference.

P. aeruginosa is an important opportunistic pathogen that can cause serious infections in individuals with a compromised immune system, particularly acute pneumonia in hospitalized patients. It also can cause infections after burns or corneal trauma, as well as chronic lung infections in cystic fibrosis patients. In each of these infections, colonization of mucosal surfaces by P. aeruginosa is the initial step. In some cases, infection remains localized to a distinct site such as the lung in cystic fibrosis patients or the eye in corneal infections. In other cases, the bacteria can disseminate; for example, in infections after burns or acute pneumonia. Therefore, it is likely that the fate of the infection depends on the site of infection and the local immune response elicited.

P. aeruginosa has been well studied for many years by many investigators. It is easy to grow and genetically manipulate. The genome of one strain has been sequenced and annotated, and the sequence analysis of additional strains is currently underway. In the case of cystic fibrosis, P. aeruginosa isolates recovered from patients during early stages of infection are similar to those most commonly found in the environment and in acute infections, typically expressing a smooth lipopolysaccharide and producing only small quantities of alginate. Chronic, respiratory-colonizing strains, on the other hand express a rough lipopolysaccharide, with few or no long O polysaccharide side chains, rendering them nontypable (unable to agglutinate with any typing serum), or polyagglutinable (reacting with more than one serum), and sensitive to killing by normal human serum. They are also mucoid due to the production of an exopolysaccharide called alginate. It is believed that the initial site of colonization of P. aeruginosa in the cystic fibrosis patient is the upper respiratory epithelium, thus inducing mucosal immunity to this pathogen would appear to be an ideal strategy for prevention of infection

P. aeruginosa vaccine candidates have included outer membrane proteins, secreted proteins such as exotoxin A and proteases, components of the type III secretion apparatus, extracellular proteins such as flagella, and pili, and extracellular polysaccharides such as alginate, and lipopolysaccharides. The most immunogenic of these candidates are the lipopolysaccharides. Challenges in the development of P. aeruginosa LPS vaccines have been reviewed by Pier, G. B. 2003. Promises and pitfalls of Pseudomonas aeruginosa LPS as a vaccine antigen. Carbohydr Res 338:2549-2556. Vaccines have been formed by expressing the O antigen portion of Pseudomonas aeruginosa lipopolysaccharide on an attenuated strain of Salmonella typhimurium. Hoiseth, S. K. et al. 1981. Aromatic-dependent Salmonella typhimurium are non-virulent and effective as live vaccines. Nature (London) 291 :238-239, incorporated herein by reference.

P. aeruginosa can be considered lipopolysaccharide-smooth (expressing many long O side chain antigens attached to the lipopolysaccharide core) or lipopolysaccharide-rough (expressing O antigens that are reduced in number, shortened in length, or completely missing). The lipopolysaccharide-smooth phenotype of P. aeruginosa imparts a serum-resistant phenotype to these isolates by a mechanism similar to that described for other Gram-negative bacteria: interfering with the insertion of the terminal components of complement (the membrane attack complex). Lipopolysaccharide-rough strains are sensitive to the action of normal human serum.

The serogroup-specific O antigens of P. aeruginosa are the immunodominant epitopes on the lipopolysaccharide and antibodies to these variant structures are protective. Both active and passive polyclonal and monoclonal immunotherapies based on the O antigens of P. aeruginosa lipopolysaccharide have been developed, but they have had limited success. These include vaccines derived from culture supernatants of various serogroup strains and those made from purified lipopolysaccharides. While both these types of vaccines have been shown to be protective in experimental infections, they each have the disadvantage of being toxic. The toxic lipid A portion of a lipopolysaccharide can be removed by acid hydrolysis, and the O antigens can be conjugated to protein carriers to elicit a stronger immune response. Both the conjugated vaccines and the high molecular weight polysaccharide vaccines show serogroup-specific protection; slight chemical differences among O antigens within a particular serogroup, which define the subtype, lead to development of subtype-specific immune responses. While protection is generally adequate against the strain to which the vaccine was developed, it is often inadequate against heterologous strains, even those belonging to the same serogroup (but different subtype). In addition, immunization of mice with vaccines incorporating two similar P. aeruginosa subtype epitopes has been shown to actually inhibit the production of broadly reactive antibodies as reported by Hatano, K., and Pier, G. B. 1998. Complex serology and immune response of mice to variant high-molecular-weight O polysaccharides isolated from Pseudomonas aeruginosa serogroup O2 strains. Infect and Immun 66:3719-3726.

A new strategy for the development of vaccines against P. aeruginosa and other pathogens is needed. All the lipopolysaccharide vaccines mentioned above are injected, or orally administered, and predominantly elicit circulating IgG antibodies. P. aeruginosa can be considered a mucosal pathogen since it can cause infections at almost any compromised mucosal surface. Therefore, a vaccine strategy should be directed towards producing effective immunity at mucosal sites. Such a mucosal immunizing vaccine must be well tolerated, and the issue of serogroup specificity must be addressed. Vaccination of patients at risk for P. aeruginosa infections should protect against subsequent infection. In cystic fibrosis patients, such a vaccine should decrease or eliminate the ability of the lipopolysaccharide-smooth initially infecting form of P. aeruginosa to establish, thus averting the inevitable pulmonary deterioration associated with colonization.

### SUMMARY OF THE INVENTION

Pseudomonas aeruginosa is often a mucosal pathogen from which it can disseminate to infect the bloodstream. It has been suggested that infection could be prevented if colonization of mucosal surfaces could be interrupted. Immunologic strategies that interrupt mucosal colonization could provide an important approach to immunotherapy. However, the immunologic mechanisms that prevent or limit bacterial colonization of a mucosal surface have been defined mostly on the basis of speculation rather than actual experimentation.

The present invention provides an attenuated Salmonella strain expressing a lipopolysaccharide O antigen from a suitable pathogen, preferably Pseudomonas aeruginosa, and the use of the same for passive immunization and as a vaccine to promote sterile immunity to the pathogen, e.g., P. aeruginosa, via intranasal vaccination. The present invention results in sterile immunity to P. aeruginosa pneumonia induced by intranasal vaccination. Accordingly, in one embodiment, the present invention is directed to a unique intranasal route of treatment and immunization by the delivery of relevant heterologous polysaccharide antigens via a live, attenuated Salmonella strain.

The advantages of the intranasal route are the ease of delivery as well as the ability to promote a robust immune response in the respiratory tract, one of the most common sites for infection.

### DESCRIPTION OF THE DRAWINGS

In the drawings:

FIG. 1 graphically illustrates IgG subtype response of sera from SL3261(pLPS2)-vaccinated mice after intraperitoneal, oral, or intransal immunization;

FIG. 2 graphically illustrates broncholalveolar lavages [BAL] of oral (A) and intraperitoneal (B) inoculated mice;

FIG. 3 graphically illustrates published survival rates for orally (A and B) and intraperitoneal (C) vaccinated animals after intranasal challenge with P. aeruginosa strain 9882-80;

FIG. 4 graphically illustrates Anti-Pseudomonas specific antibody analysis of broncholaveolar lavage (A and B) and nasal washes (C);

FIG. 5 graphically illustrates survival studies for mice infected with three different doses of P. aeruginosa, (A—6 times LD₅₀), (B—12 times LD₅₀), (C—3 times LD₅₀);

FIG. 6 graphically illustrates protection from P. aeruginosa corneal infection in mice following intranasal vaccination;

FIG. 7 graphically represents protection from P. aeruginosa burn infection following intranasal vaccination; and

FIG. 8 graphically represents passive protection studies of intransal vaccine immunized mice given antisera. (A—diluted antisera given immediately before intranasal infection with PA103), (B—whole antisera given 6 hours post infection with PA 103), (C—whole antisera given 6 hours post infection with the serogroup O6 strain, 6294), and (D—diluted antisera immediately before intransal infection with PA103).

### DEFINITIONS

In describing and claiming the invention, the following terminology will be used in accordance with the definitions set forth below.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, are reduced.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

The term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopia for use in animals, including humans.

As used herein, the term “treating” includes prophylaxis of the specific disorder or condition, or alleviation of the symptoms associated with a specific disorder or condition and/or preventing or eliminating said symptoms via passive immunization.

The term “additional ingredients” in connection with formulations for delivery of vaccines includes, but is not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

### DESCRIPTION OF THE PREFERRED EMBODIMENTS

Methods used to express P aeruginosa O antigens in a heterologous system such as Salmonella are well known and do not constitute a part of this invention. To express P. aeruginosa O antigens in a heterologous system such as Salmonella, the genes encoding the enzymes for their biosynthesis must be cloned. Fortunately, the genes encoding the enzymes for the O antigen are often clustered in the chromosome and can be isolated on large DNA fragments. The sequences encoding the genes for P. aeruginosa O antigen biosynthesis and assembly have been studied in detail for serogroups O5, O6 and O11 and reported by Burrows et al 1996. Molecular characterization of the Pseudomonas aeruginosa serotype O5 (PAO1) B-band LPS gene cluster. Molec Microbiol 22:481-495; Belanger et al 1999. Functional analysis of genes responsible for the synthesis of the B-band O antigen of Pseudomonas aeruginosa serotype O6 lipopolysaccharide. Microbiol 145:3505-3521; and Dean et al 1999. Characterization of the serogroup O11 O-antigen locus of Pseudomonas aeruginosa PA103. J Bacteriol 181:4275-4284, respectively. Each of the aforesaid references are incorporated herein for their teachings of the method used to express P aeruginosa O antigens in a heterologous system.

To determine the effectiveness of intranasal administration of the P. aeruginosa antigen, DNA from the cytotoxic serogroup O11 P. aeruginosa strain PA103 was cloned into the cosmid vector pLAFR1 by complementing the LPS-rough serum-sensitive phenotype of a number of CF isolates. This clone, pLPS2, encoded all the genes for expression of P. aeruginosa O11 O antigen, based on the ability of Escherichia coli HB101 containing pLPS2 to express P. aeruginosa serogroup O11 O antigen. SDS-PAGE analysis demonstrated that E. coli HB101 containing the vector, pLAFR1, expressed a rough lipopolysaccharide (as does E. coli HB101 without a plasmid), whereas lipopolysaccharide from E. coli HB101(pLPS2) produced a ladder-like pattern typical of a smooth lipopolysaccharide. Antibodies to the lipopolysaccharide of P. aeruginosa serogroup O11 reacted on Western blots with homologous lipopolysaccharide and with E. coli HB101 (pLPS2) LPS, but not with lipopolysaccharides from E. coli HB101(pLAFR1). This work is reported in Goldberg et al 1992. Cloning and surface expression of Pseudomonas aeruginosa O antigen in Escherichia coli. Proc Natl Acad Sci, USA. 89:10716-10720 incorporated herein by reference.

To investigate a potential means of eliciting protective immunity against P. aeruginosa serogroup O11, advantage was taken of the expression of this O antigen on the surface of E. coli and used this pLPS2-containing strain to study the immune response specific to the lipopolysaccharide of P. aeruginosa serogroup O11. Mice were fed 10⁹ CFU of either E. coli strain in 100 μl of 2% sucrose/2% bicarbonate once a week for 4 weeks. Mice fed E. coli HB101(pLPS2) produced serum IgG and both serum and mucosal IgA antibodies to P. aeruginosa serogroup O11 O antigens. No response was observed in samples from mice fed E. coli HB101(pLAFR1).

Both the expression of the P. aeruginosa O side chain antigens by E. coli and the immune response elicited to E. coli HB101(pLPS2) suggested that similar expression might be possible by the closely related enteric bacteria Salmonella, which has been used as a vaccine candidate. The pLPS2 was transferred to four different strains of Salmonella: the wild-type S. typhimurium strain CS019, the galE mutant S. typhimurium strain LB5010. S. typhi strain Ty21a, the prototype human vaccine strain, and the aroA mutant S. typhimurium strain SL3261.

All four pLPS2-containing strains expressed P. aeruginosa serogroup O 11 O antigens. There was some concern that expression of P. aeruginosa O antigen might restore virulence to the attenuated strains. However, no increase in virulence was seen in mice fed the S. typhimurium aroA mutant SL3261 (pLPS2) as compared to those fed L3261 (pLAFR1). In fact, no mice died following oral challenge of up to 10¹⁰ CFU/mouse of either SL3261 strain. These findings indicate that expression of P. aeruginosa serogroup O11 O antigen does not increase the virulence in an attenuated aroA background. Thus, preliminary animal experiments with the S. typhimurium aroA mutant, SL3261 were initiated. Fortunately, for any studies using S. typhi Ty21a, restored virulence is not likely to be a problem, since this strain has been shown that other, as yet uncharacterized, mutations account for its reduced virulence in humans.

Since expression of P. aeruginosa serogroup O11 antigen in the attenuated S. typhimurium strain was observed, the immune response and clearance of a serogroup O11 strain from the gastrointestinal (GI) tract after oral immunization was tested. For oral immunization with recombinant Salmonella strains, BALB/c mice were fed 100 μl of 2% sucrose/2% bicarbonate containing 10⁹ CFU of either SL3261 (pLPS2) or SL3261 (PLAFR1): Mice received 3-4 doses at intervals of 3-4 days. Gut colonization of mice with S. typhimurium SL3621(pLPS2) elicited serum and mucosal IgA antibodies to P. aeruginosa serogroup O11 O antigen, while mice colonized with SL3621(pLAFR1) did not. Mice that were colonized with either S. typhimurium strain were cleared of indigenous facultative flora by administration of streptomycin for 5 days. For challenge, the non-cytotoxic serogroup O11 P. aeruginosa strain, 9882-80, was added to the drinking water of the mice at a concentration of 10⁷ CFU/ml. The extent of bacterial clearance was examined by determining the CFU of P. aeruginosa/gram of feces. Reduced persistence of the P. aeruginosa challenge strain was observed in mice orally immunized with S. typhimurium SL3261(pLPS2) compared with those orally immunized with S. typhimurium SL3261(pLAFR1). These results show that a recombinant strain of S. typhimurium expressing P. aeruginosa O antigen can be safely administered orally and can elicit immunity that can protect mice against subsequent GI colonization by an LPS-homologous P. aeruginosa strain. These results are reported in Pier et al 1995. Clearance of Pseudomonas aeruginosa from the murine gastrointestinal tract is effectively mediated by O-antigen-specific circulating antibodies. Infect Immun 63:2818-2825, incorporated herein by reference.

The findings that SL3261 (pLPS2) could induce a specific serum and mucosal IgA and serum IgG response to recombinant P. aeruginosa O antigens after oral administration suggests that this route of immunization may provide protection at a more relevant site such as the lung, through the effect of the common mucosal immune system. However, it was not know how oral (mucosal) immunization would compare with intraperitoneal (systemic) immunization with this vaccine in terms of generating a specific immune response and protection against P. aeruginosa acute pneumonia.

For oral vaccination, BALB/c mice were fed 100 μl of either PBS. SL3261(pLAFR1) (vector) or SL3261(pLPS2) (vaccine) (1-5×10⁹ CFU), by intragastric lavage. Oral inoculation was repeated once per week for a total of four weeks. For intraperitoneal vaccination, BALB/c mice were inoculated with a single dose of either PBS or one of the Salmonella strains (10⁶ CFU). Serum samples, taken one week after each booster, showed increased levels of anti-Pseudomonas-specific IgG and lgA in vaccine-inoculated animals after each oral vaccination, with the highest IgG and IgA titers observed one week after the final booster. The same trend was observed in intraperitoneal vaccinated mice, with animals receiving the vaccine yielding significant Pseudomonas-specific serum IgG when compared to the controls. Sera from intraperitoneal inoculated mice had significantly greater Pseudomonas-specific IgG titers, approximately two-fold higher than seen in orally vaccine-inoculated mice. Pooled sera from orally vaccinated animals were reactive to four additional serogroup O11 strains by ELISA. Similar specificity was observed with pooled sera from intraperitoneal vaccinated mice, which had high IgG titers to all O11 strains tested. Serum IgA reactivity in pooled antisera was only seen in orally vaccinated animals: Pseudomonas-specific serum IgA was not detected above pre-immune levels in intraperitoneal vaccinated animals. These results are reported in DiGiandomenico et al 2004. Oral vaccination of BALB/c mice with Salmonella enterica serovar Typhimurium expressing Pseudomonas aeruginosa O antigen promotes increased survival in an acute fatal pneumonia model. Infect Immun 72:7012-7021, incorporated herein by reference.

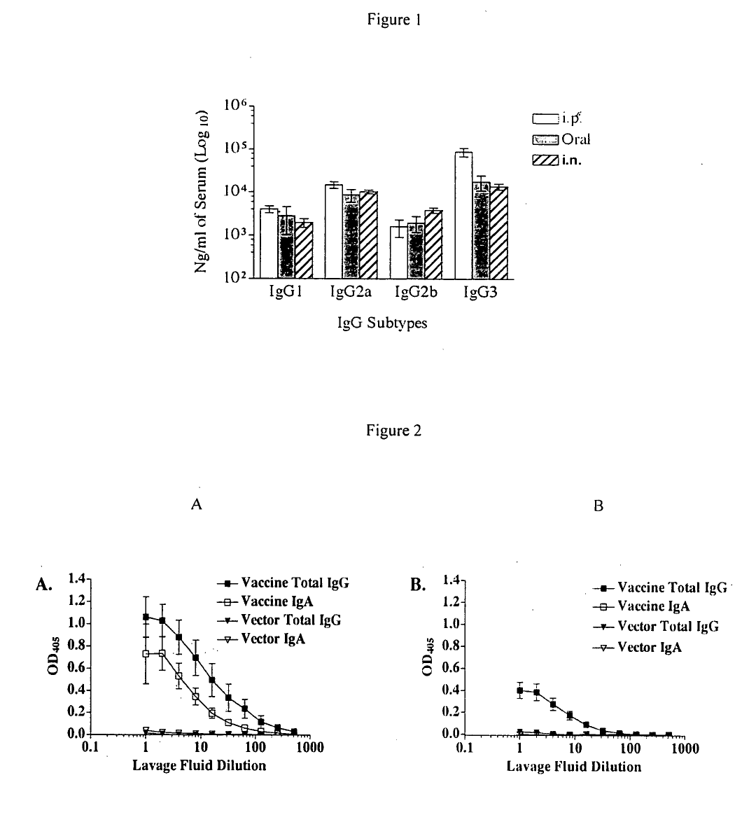

The IgG subtype response was determined for both oral and intraperitoneal vaccinated animals. Orally immunized mice produced similar levels of serum IgG2a and IgG3 and these antibody levels were significantly higher when compared to either IgG1 or IgG2b levels. In contrast, intraperitoneal vaccination induced greater levels of IgG3 as compared to all IgG subtypes, and this level was approximately four-fold greater than the level of IgG3 in oral-immunized mice as shown by FIG. 1 of the drawings where the bars indicate the level of each IgG subtype in log₁₀ calculated to mg per ml of serum based on standard curves generated for secondary antibody reactivity to each IgG subtype. Error bars represent the standard deviation. In addition, IgG2a antibody levels were greater in intraperitoneal vaccinated animals as compared to IgG1 and IgG2b and approximately two fold higher than the level of IgG2a seen after oral vaccination. These results indicate that oral immunization produces lower levels of serum IgG antibodies with a distinct ratio of IgG subclasses compared to intraperitoneal immunization.

To determine whether the differences in the antibody response correlated with the ability to protect against P. aeruginosa acute pneumonia, oral and intraperitoneal vaccinated mice were challenged by intranasal infection with the non-cytotoxic serogroup O11 P. aeruginosa strain, 9882-80 at a dose of ˜1.1×10⁸ CFU (˜6 times the LD₅₀). In orally immunized mice, there was a significant increase in survival post infection compared to mice that received PBS or the vector. Interestingly, challenge of intraperitoneal vaccinated nice with 9882-80 failed to show a difference in survival of vaccine-immunized mice when compared to the PBS and vector controls, even though the serum IgG antibodies in these animals were significantly greater than the levels seen in orally vaccinated mice.

To identify if protection could be extended to P. aeruginosa serogroup heterologous strains and to examine whether the cross-reactive epitopes contributed to protection in this model, orally vaccinated mice were infected with strain 6294 (serogroup O6) at a challenge dose approximately six-times the LD₅₀ that was calculated for this strain. No difference in survival was noted between vector and vaccine-inoculated mice, supporting previous results that the vaccine strain induces a serogroup O11-specific response and suggesting that any cross-reactive epitopes are not protective. Altogether these results suggest that the vaccine strain induces serogroup O11-specific protection and is more efficacious when delivered orally.

Opsonophagocytic killing assays are often considered good in vitro correlates of protection. It was found that sera from mice immunized with the vaccine by the intraperitoneal route showed higher levels of oposonophagocytic killing than sera from orally vaccinated mice. In both cases, the killing was shown to be serogroup-specific, as no killing was observed with the serogroup O6 strain. These findings suggest that intraperitoneal immunization can elicit protective systemic antibodies but these are not in the correct location to provide protection against an intransal infection.

With evidence supporting that oral, rather than intraperitoneal, immunization provides increased survival in the P. aeruginosa pneumonia model, it was desirable to ascertain whether or not differences in the antibody profiles existed at the site of infection after either vaccination. To accomplish this, broncholaveolar ravages (BAL) were performed on mice after oral and intraperitoneal inoculation. In mice that received the oral vaccine, high levels of total IgG and IgA in BAL fluid were observed, suggesting that the gut-associated lymphoid tissue (GALT) is sufficiently activated by the vaccine allowing antibody secreting B-cells to localize to the respiratory lymphoid tissue. Only low-level reactivity for total IgG was seen in the BAL of intraperitoneal vaccinated animals, with no evidence of Pseudomonas-specific IgA present. These results are shown in FIGS. 2A and 2B. The lack of IgA in pulmonary secretions from intraperitoneal vaccinated mice was not unexpected since this antibody was similarly not observed in the serum from these animals. Although these results clearly do not rule out a function of IgG-mediated protection in oral vaccinated mice, they do suggest that IgA may have a protective role. Work by other groups has shown that intraperitoneal administration of attenuated Salmonella organisms produce potent systemic antibody responses, but lack mucosal stimulation, as determined by the absence of an IgA response in serum and mucosal secretions as confirmed by the above results.

From the above, it was concluded that oral immunization with the recombinant SL3261 expressing P. aeruginosa O antigen was more effective in protection against acute P. aeruginosa pneumonia than intraperitoneal immunization. This suggests that the specific immune response and location of the antibodies generated by oral immunization promote this increased survival.

Having successfully shown that oral delivery of the vaccine initiates mucosal and systemic immune activation that mediates increased survival to Pseudomonas acute pneumonia, it was desired to determine if local administration of the vaccine was immunogenic and could perhaps provide even greater protection. Mice were intranasal vaccinated by placing 10 μl of PBS or bacterial inoculum onto each nostril at a dose of 1×10⁷ CFU (total of 20 μl/mouse), followed by a booster after 14 days. Analysis of serum immunogenicity was determined using ELISA with plates coated with P. aeruginosa PA 103 (serogroup O11). Sera isolated from animals that received the vaccine exhibited a robust anti-Pseudomonas total IgG and IgA antibody response as compared to sera from vector or PBS treated control animals. Moreover, pooled sera from intransal vaccinated animals were reactive to four additional serogroup O11 strains, suggesting that intransal delivery of the vaccine produces a broad serogroup O11 response.

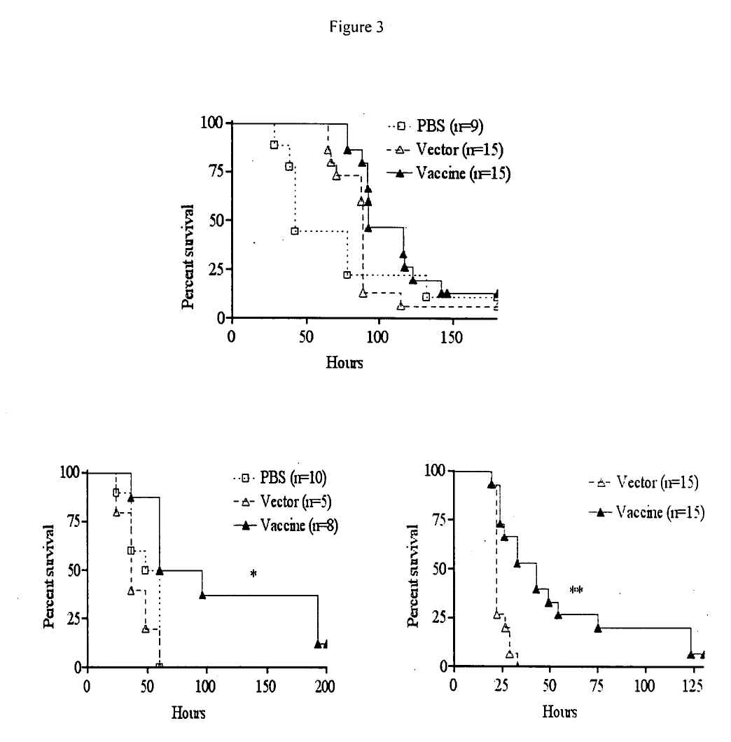

The serum IgG subtype response was determined for in mice that received intransal vaccine. Results are represented in comparison to previously published data from oral and intraperitoneal vaccinated animals and shown in reproduced FIG. 3 of the drawings from DiGiandomenico et al, supra where results are represented as Kaplan-Meier survival curves, and differences in survival were calculated by the log-rank test. Similar to what was seen following oral and intraperitoneal inoculation, intranasal vaccine delivery induced a distinct ratio of IgG subclasses. Of particular note, and similar to that observed for oral immunization, the level of IgG2a produced was much higher than the level of IgG1, a response consistent with the production of a Th1-induced antibody response. It was also noted that the levels of IgG2a and IgG3 were similar to one another and were significantly higher when compared individually to either IgG1 or IgG2b. These results suggest that intransal vaccination with the subject recombinant vaccine strain induces a broad serogroup O11 specific P. aeruginosa antibody response that is indicative of the Th1 induced pathway.

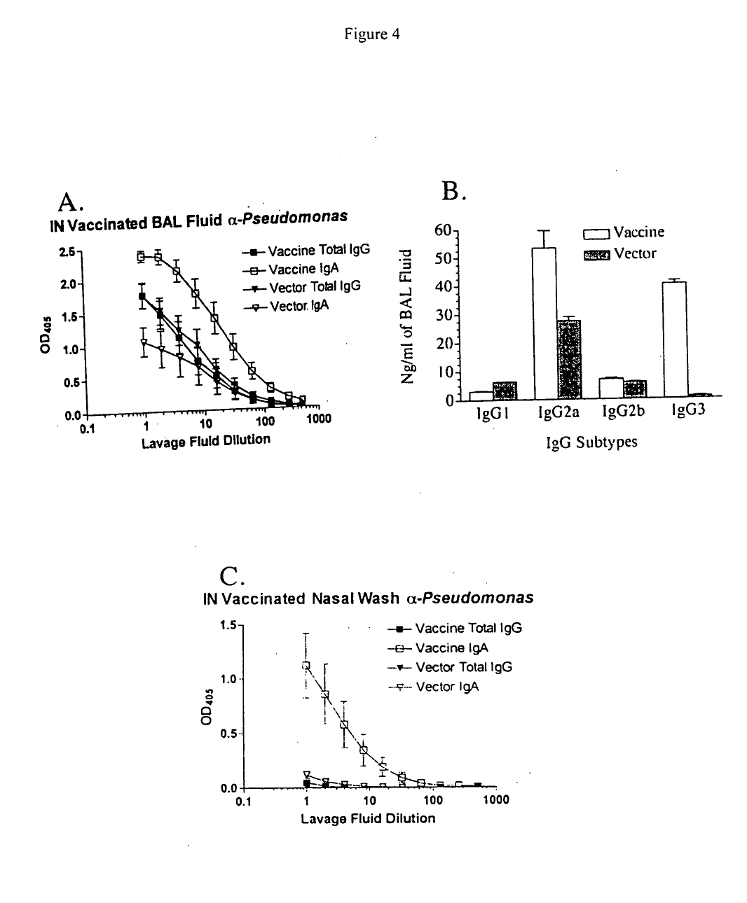

Having identified that intranasal vaccination stimulates a robust systemic antibody response to P. aeruginosa, the relative level of antibodies isolated from mucosal lavage fluid from these animals was examined. BAL fluid of vaccinated mice revealed high levels of total IgG and IgA present in the lower respiratory tract with the level of IgA reactivity nearly exceeding IgG by two-fold as shown by FIG. 4A. No detectable difference in IgG reactivity between vector and vaccine when probing with an anti-lgG secondary antibody was observed. To decipher Pseudomonas-specific IgG levels from background, BAL fluid was examined for the presence of anti-P. aeruginosa specific IgG subtypes. The BAL of animals receiving the vaccine had significantly higher levels of IgG2a and IgG3 as compared to background levels seen for mice receiving the vector. In addition, no difference in the level of IgG1 and IgG2b was observed between mice receiving vector or vaccine as shown by FIG. 4B. These results are consistent with the serum IgG subtype response and further support the evidence of a Th1-skewed immune response induced by the vaccine.

To identify the presence of Pseudomonas-specific antibodies in the upper respiratory tract (URT) of intranasal vaccinated mice, nasal washes were performed. Only Pseudomonas-specific IgA could be detected in this location with no evidence of Pseudomonas-specific IgG reactivity above background levels as shown by FIG. 4C. In addition, Pseudomonas-specific IgG and IgA were detected in the saliva of intranasal vaccinated animals. Altogether, these results indicate that intranasal immunization with the vaccine strain induces potent systemic and mucosal immunity to P. aeruginosa serogroup O11 O antigen.

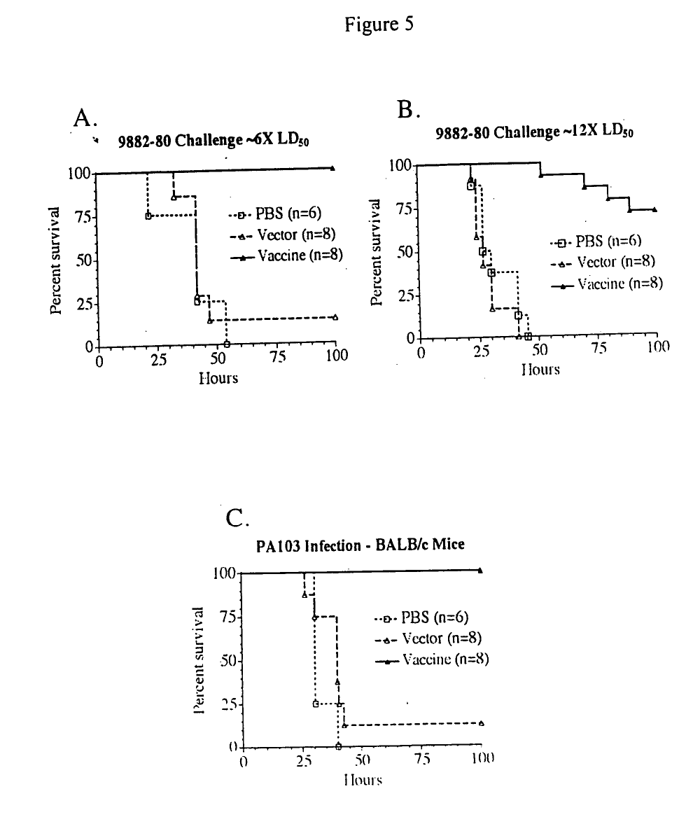

To test the ability of the vaccine to confer protection to P. aeruginosa acute pneumonia, mice were challenged by intranasal infection with 2 different serogroup O11 strains, 9882-80 or PA103, which are non-cytotoxic and cytotoxic, respectively. Vaccinated animals were challenged with 9882-80 at a dose that was six-times the LD₅₀ for this strain and complete protection was observed only in mice that received the vaccine as illustrated in FIG. 5A of the drawings. In a second survival study using 9882-80 as the challenge organism, vaccinated mice were infected with approximately twelve-times the LD₅₀; seventy-two percent of the animals that received the vaccine survived, while all PBS and vector control animals succumbed to infection as shown by FIG. 5B. Since protection to 9882-80 was strong, a decision was made to examine whether the vaccine could provide the same level of protection to PA103, which was determined to be approximately 200 times more virulent than 9882-80. It was found that vaccine inoculated mice were completely protected from PA103 infection at three times the LD₅₀ and showed no signs of morbidity throughout the experiment as shown by FIG. 5C. In addition to BALB/c mice, it was determined that intranasal immunization protects against subsequent infection by PA103 in C57BL/6, C3H/HeN, CF-1, and B6129SF2/J mice. Among this group, C3H, C57BL/6, and BALB/c were previously shown to be susceptible to intranasal infection with P. aeruginosa. The findings indicate that each of these mouse strains, regardless of whether they are Th1- or Th2-dominant responders, can be protected from P. aeruginosa infection by the subject vaccine construct.

To follow in vivo infection, a luminescent strain of PA103, PA103.lux was constructed. This was accomplished by integrating the lux gene cluster, under the transcriptional control of the lac promoter, into a non-essential region of the PA103 genome. The detection limit of PA103.lux was determined to be approximately 10⁵ organisms when tested in serial dilutions on microtiter plates. Integration of the lux gene cluster into the PA103 genome had no apparent effect on the virulence of the strain. To monitor bacterial dissemination in real-time, naive, vector, and vaccine-immunized mice were infected by intranasal administration (5×10⁵-1×10⁶) with PA103.lux and imaged at 12 and 24 hours post-infection using the Xenogen IVIS Imaging system. In both naive and vector-immunized mice, strong signals could be detected in the URT, or nasopharynx, and the lungs. In addition, signals were also detected from the GI tract. However, there was no detectable evidence of luminescence in vaccine-immunized mice at either time-point, suggesting that these animals efficiently removed the infecting strain. Nasal lavages from naïve and vector-vaccinated animals confirmed the high level on PA103.lux colonization of the URT at both 12 and 24 hours, and that viable CFU in nasal lavages from vaccine-immunized animals were barely detectable at 12 hours and absent at 24 hours. To confirm the presence of PA103.lux in imaged animals, mice were euthanized and their lung, spleen, and liver were harvested. homogenized, and plated for viable cell counts. No difference in PA103.lux CFU was noted in the lungs of naïve or vector-immunized animals at 12 hours post-infection, though at 24 hours a few of the vector-immunized animals had already succumbed to infection and were unable to be analyzed. Similar results were noted in the viable CFU counts in spleen and liver homogenized tissues from naïve and vector-immunized mice, with bacterial dissemination apparent. Consistent with the IVIS images, vaccine-immunized animals had little to no bacteria in their lungs at 12 hours post-infection with no signs of dissemination to the spleen or liver. By 24 hours, no evidence of PA103.lux was detected in any of the tissues. Altogether, these results confirm that vaccine-immunized mice efficiently clear Pseudomonas after intranasal infection, most likely by antibody and complement-mediate opsonophagocytosis.

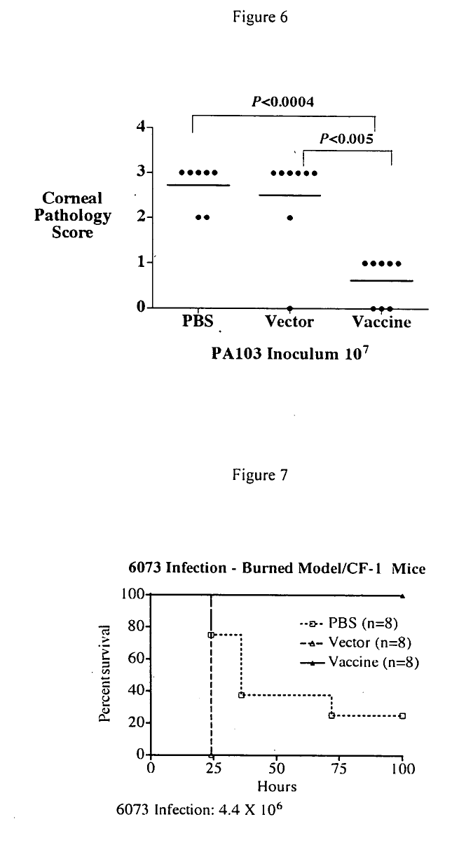

Since dramatic protection against acute P. aeruginosa pneumonia was observed using intranasal immunization, an effort was undertaken to determine if the vaccine would protect against different types of P. aeruginosa infections. For the corneal infection model, C3H/HeN mice were immunized as previously described with either PBS, S. typhimurium SL3261(pLAFR1) (vector), or SL3261(pLPS2) (vaccine) (1×10⁷ CFU in 20 μl), twice at 0 and 14 days. Three weeks later, the mice were anesthetized with xylazine and ketamine and 3 l-mm scratches were made in the cornea and superficial stroma of one eye of each mouse with a 27-gauge needle. Mice were infected with 1×10⁶ CFU of strain PA103 in 5 μl, a dose previously shown to cause severe infections. Mice were monitored for 2 days and pathology was scored. FIG. 6 shows the results. Mice immunized with PBS or the vector showed significant pathology that was similar between the two groups. Most importantly, the vaccine-immunized mice showed significantly less pathology.

For the burn infection model, serogroup O11 strains of P. aeruginosa are not among those generally used for assessing virulence. Therefore, a determination was made of the LD₅₀ of a number of different serogroup O11 strains after burns. Compared to the typical test strain that results in an LD₅₀ at doses as low as 10³ after burns, the six serogroup O11 strains (including PA103) each required doses of >10⁶ to achieve an LD₅₀. The strain with the lowest LD₅₀, 6073, was used for the subsequent challenge experiment.

CF-1 mice were intranasal immunized with PBS, the vector, or vaccine in a single dose (1×10⁷ CFU in 20 μl). After 3 weeks, for infection after burns, the hair was clipped from the mice and they were anesthetized with isoflurane. A flame resistant card was pressed against the shaved area; the exposed area was covered with 0.5 ml of ethanol, ignited, and allowed to burn for 10 seconds. The mice were immediately given 0.5 ml of saline for fluid resuscitation. The procedure produces a 15% total body surface area non-lethal third degree burn. Strain 6073 was injected subcutaneously at the burn site with 4.4×10⁶ CFU. As shown in FIG. 7, mice that received the vaccine survived the infection, while all the mice receiving the vector succumbed by 25 hours. The PBS-treated in mice survived slightly longer than the vector-treated mice but this was not statistically different than vector-immunized mice.

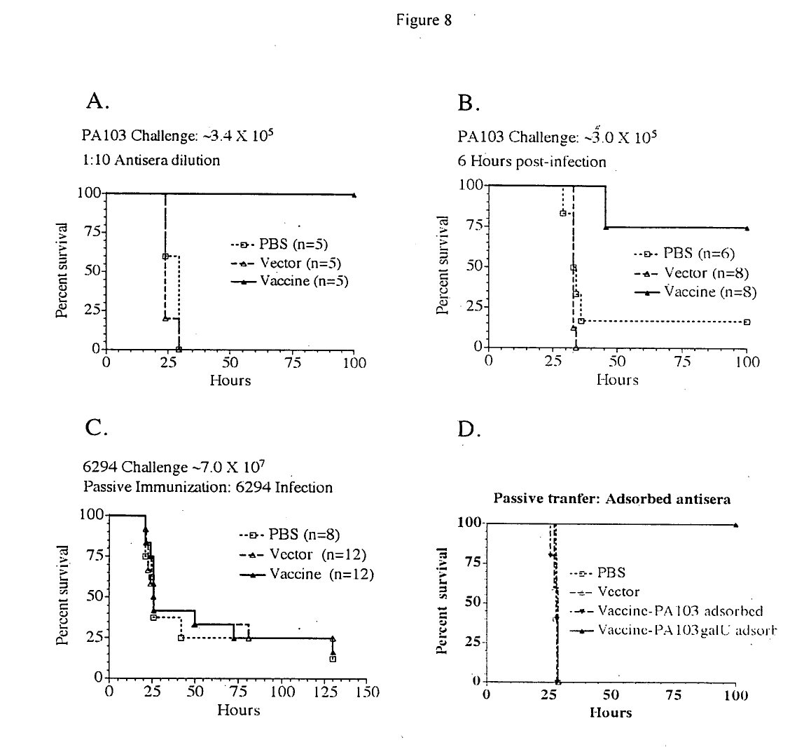

To confirm that serum serogroup O11-specific antibodies conferred protection to P. aeruginosa pneumonia, PA103 infections were performed on naive mice prior to or after intranasal passive transfer of PBS, vector, or vaccine-immunized antisera. In the initial protection study, antisera were transferred to mice before infection with PA103. Mice that received diluted vaccine antisera (1:10) immediately after infection were completely protected from challenge doses of 3.4×10⁵ of PA103 with no signs of morbidity as shown in FIG. 8A. To identify if animals could be rescued from PA103-induced pneumonia, antisera was transferred to mice six-hours post-infection. Seventy-five percent of mice receiving vaccine antisera were rescued from infection, while nearly all of the controls succumbed as shown by FIG. 8B. Passive transfer of antisera to mice was tested to determine if it could provide protection to LPS-heterologous strain 6294. No difference in survival was noted in mice that received PBS, vector, or vaccine antisera, suggesting that protection is primarily due to serogroup O11-specific antibodies induced by the vaccine. This is illustrated in FIG. 8C of the drawings. To validate this finding, diluted vaccine antisera (1:10) was adsorbed with either PA103 or PA103galU, an O-antigen deficient mutant, prior to transfer to naïve animals. Complete protection was only seen in infected mice receiving PA103galU adsorbed antisera, which still contained O-antigen-specific antibody as illustrated in FIG. 8D. These results confirm that protection in this model is primarily mediated by serogroup-O11 specific antibodies.

A description of materials and methods used to obtain the above results follows.

Bacterial Strains: Bacterial strains and plasmids are listed in Table 1 along with their descriptions and sources.

TABLE I

| Salmonella | |
|---|---|
| SL3261/pLAFR1 | ΔaroA derivative of SL1344 containing |
| | a control cosmid vector |
| SL3261/pLPS2 | ΔaroA derivative of SL1344 harboring a plasmid |
| | containing the PA103 serogroup O11 |
| | O-antigen gene cluster |
| P. aeruginoisa | |
| PA103 | Wild-type strain; serogroup O11 |
| PA 103 (galU) | O-antigen deficient serogroup O11 derivative |
| 9882-80 | Clinical isolate; serogroup O11 |
| 6073 | Clinical isolate; serogroup O11 |
| 6294 | Clinical isolate; serogroup O6 |

Preparation of Bacterial Strains: The serogroup O11 gene locus is contained within cosmid vector pLAFR1, referred to here as pLPS2. Constructs SL3261/pLAFR1 and SL3261/pLPS2 are referred to here as the vector and vaccine strains, respectively. Salmonella vector and vaccine strains were inoculated into Luria-Bertani broth containing tetracycline at 10 μg/ml and grown to an optical density at 650 nm (OD₆₅₀) of 0.5. Cells were harvested by centrifugation at 3,100×g for 20 min at 4° C. and resuspended in phosphate buffered saline supplemented with 3% sodium bicarbonate and 2% sucrose for oral vaccination. Prior to administration to animals, bacterial cells were adjusted spectrophotometrically and plated on tryptic soy agar (TSA) to determine viable cell counts.

Oral and intranasal i.p. vaccinations. Six- to 8-week-old BALB/c mice (Jackson Laboratories, Bar Harbor, Me., or Harlan Sprague-Dawley Farms, Chicago, Ill.) were housed under pathogen-free conditions and fed autoclaved rodent feed and acid-free water. For oral vaccination, mice were fed 100 μ/l of either PBS or the Salmonella vector and vaccine strains (1×10⁹ to 5×10⁹ CFU) by intragastric lavage with a 20/25-mm feeding needle (Popper and Sons, Inc., New Hyde Park, N.Y.) attached to a 1.0-ml Becton Dickinson (BD) Luer-Lock syringe (Fisher Scientific, Pittsburgh, Pa.). Oral inoculation was repeated once per week for a total of 4 weeks. For intranasal vaccination, mice were vaccinated by placing 10 μl of PBS or bacterial inoculum onto each nostril at a dose of 1×10⁷ CFU (total of 20 μl/mouse), followed by a booster after 14 days.

Survival studies. Challenge experiments were performed with P. aeruginosa strains 9882-80 (serogroup O11), PA 103 (serogroup O11), and 6294 (serogroup O6). The appropriate strains were inoculated onto TSA and grown for a maximum of 12 h at 37° C. Cells were resuspended in PBS to an OD₆₅₀ of 0.5 and diluted to the appropriate concentration to obtain the desired challenge dose in 20 μl. Prior to challenge, mice were anesthetized by i.p. injection of 0.2 ml of freshly prepared filter sterilized ketamine (6.7 mg/ml) and xylazine (1.3 mg/ml) in 0.9% saline. Once the mice were anesthetized. 10 μl of the bacterial inoculum was placed into each nostril (total of 20 μL/mouse). The mice were observed carefully for morbidity and mortality for up to 1 week.

Collection of BAL fluid. Mice were euthanized by anesthetic overdosing with 0.5 ml of freshly prepared filter-sterilized ketamine and xylazine injected i.p. The tracheas of these animals were exposed by standard procedures and cannulated with a 20-gauge, 1.16-in., 1.1-by 30-mm BD tracheal cannula attached to a Luer-Lock syringe needle. One milliliter of PBS was introduced into the lungs via the tracheal cannula and carefully extracted. This procedure was repeated once more, for a total of 2 ml of PBS for each lavage. The broncholaveolar lavage (BAL) fluid was analyzed immediately by an indirect enzyme-linked immunosorbent assay (ELISA) for Pseudomonas-specific antibodies.

Serum collection. Blood samples were collected from the tail vein of each mouse after warming with a heat lamp. The samples were allowed to stand at room temperature for 4 hours and then were incubated overnight at 4° C. Serum was collected by centrifugation at 1,700×g for 10 min and then was stored at −80° C. until use. All serum samples were diluted in PBS supplemented with 1% bovine serum albumin (PBS-B) prior to use in ELISAs.

Indirect and competition ELISAs. The optimal concentrations of whole organisms (either P. aeruginosa or SL3261) for use as coating antigens were determined by criss-cross serial dilution with P. aeruginosa serogroup O11 (Accurate Chemical, Westbury, N.Y.)- or Salmonella O antiserum factor 4 (Difco Laboratories, Detroit, Mich.)-specific polyclonal antibodies. P. aeruginosa organisms or SL3261 were inoculated onto TSA and grown for a maximum of 12 h at 37° C. Bacteria were suspended in 0.1 M sodium phosphate buffer to an OD₆₅₀ of 0.5, diluted 1:2, and used to coat Immulon 2 HB ELISA plates (Thermo Lab Systems, Franklin, Mass.). The plates were incubated overnight at 4° C. to allow for sufficient antigen coating, washed with PBS supplemented with 0.05% Tween 20 (PBS-T), and blocked by 1 h of incubation at room temperature with PBS-B. After a second wash with PBS-T, the ELISA plates were stored at 4° C. until use.

For antibody titer determinations, serum and BAL samples were serially diluted in PBS-B, and 100 μl was placed into each well on antigen-coated plates in duplicate. After overnight incubation at 4° C., the plates were washed three times with PBS-T and dried. Secondary antibodies (anti-mouse total IgG, IgG1, IgG2a, IgG2b, IgG3, or IgA conjugated to alkaline phosphatase [Southern Biotechnology Associates, Inc. Birmingham. Ala.]) were diluted 1:5,000 in PBS-B, and 100 82 l was added to appropriate wells and incubated at 37° C. for 90 min. After incubation, the plates were carefully washed again three times with PBS-T and dried. Next, 200 μl of the substrate, consisting of 4-nitrophenyl phosphate disodium salt hexahydrate (PNPP) (Sigma Chemical Co., St. Louis, Mo.) diluted to 1 mg/ml in PNPP substrate solution (10% diethanolamine, 25 μM MgCl₂), was added to each well and incubated for 30 min in darkness at room temperature. PNPP hydrolysis was terminated by the addition of 50 μl of 3 M NaOH to each well, the plates were examined at OD₄₀₅ by use of a Molecular Devices Thermo microplate reader, and the data were displayed by use of SOFTmax Pro version 1.1 software.

For IgG subtype quantification, a mouse immunoglobulin standard panel (100 μg/ml; Southern Biotechnology Associates) was serially diluted and used to coat ELISA plates. The appropriate secondary antibody was used, and the ELISA was carried out as described above. IgG subtype quantification for serum samples was based on standard curves that were designed for each antibody isotype by use of GraphPad (San Diego, Calif.) Prism version 4 software. For the competition ELISA, plates coated with PA103 whole organisms were used. The ELISA was performed as described above but with the following exceptions. To a single constant 1:5,000 dilution of pooled sera from vaccine inoculated mice were added increasing amounts (0 to 5,000 ng) of free purified serogroup O11 LPS. The competition ELISA was performed twice in triplicate.

SDS-PAGE and Western immunoblotting. Whole-cell lysates of P. aeruginosa and Salmonella organisms were separated by sodium dodecyl sulfate (SDS)-polyacrylamide gel electrophoresis (PAGE) with a Novex X-Cell Surelock minicell system (Invitrogen, Carlsbad, Calif.). Tris-bis-polyacrylamide gels (12.5%) were cast in 1.0-mm Invitrogen cassettes. After PAGE separation was completed, lysates were electroblotted onto Trans-Blot 0.2-μm-pore-size pure nitrocellulose membranes (Bio-Rad Laboratories, Hercules, Calif.) by use of a Bio-Rad Mini Trans-Blot electrophoretic transfer cell. Membranes were blocked and then probed with Pseudomonas serogroup O11- or Salmonella O antiserum factor 4-specific polyclonal antibodies, followed by incubation with anti-rabbit secondary antibodies conjugated to alkaline phosphatase (Sigma). Reactions were visualized by the addition of Sigma fast 5-bromo-4-chloro-3-indolylphosphate-nitroblue tetrazolium.

Opsonophagocytosis assay. Briefly, the assay consists of four components: freshly prepared dextran-purified white blood cells (obtained from 30 ml of human blood by use of an 18-gauge, 1-in. hypodermic needle [SIMS Portex Inc., Keene, N.H.] attached to a 30-mil BD syringe), baby rabbit serum as a complement source (Accurate Chemical), pre- and postimmunization sera at various dilutions, and the target P. aeruginosa strains. Prior to use in this study, all target strains were confirmed for their ability to resist serum killing by repeated incubations with normal human serum and passages in Luria-Bertani broth. All four components were incubated together at 37° C. with slight agitation for 90 min, followed by the determination of viable cell counts on TSA. The percentage of killing was determined by comparing the number of colonies isolated from diluted. postimmunization antisera to the number of colonies obtained from preimmunization sera at the same dilution. Under these conditions, observation of 50% killing is considered to be biologically significant and thus serves as the point at which antisera are considered positive for opsonic killing activity.

Statistical analyses. All analyses were performed by use of GraphPad Prism version 4 software. ELISA endpoint titers were calculated by linear regression of duplicate measurements of adjusted OD₄₀₅s and were expressed as the reciprocal dilution. OD₄₀₅s of preimmunized samples at the same dilution were subtracted from those of postimmunization samples. The x intercept served as the endpoint titer. Pseudomonas-specific total lgG and lgA titers were compared by using the Kruskal-Wallis U test for analysis of three groups or the Mann-Whitney U test for comparison of two groups. IgG subtype values were analyzed by use of a mixed-linear-model methodology. The natural logarithms of the IgG subtype values were modeled by use of a compound symmetric covariance structure to adjust for correlation among measurements obtained from the same mouse. For survival studies, data are presented as Kaplan-Meier survival curves and were analyzed by the log-rank test. For comparison of the numbers of viable bacteria obtained in lung homogenates from PBS-, vector-, and vaccine-inoculated animals, the data were transformed to natural logarithms and analyzed by one-way analysis of variance.

The above suggests the use of additional intranasal vaccines against select pathogens. For example, the cloning and expressing additional O antigen (polysaccharide) loci from any Gram-negative pathogen, particularly those causing serious infections including various Escherichia coli, Vibrio cholerae, Haemophilus influenzae, Moraxella catarrhalis, Neisseria meningitides, and Bordetella pertussis, as well as biodefense agents including Yersinia, Burkholderia, Coxiella, Brucella, Shigella, Salmonella, Campylobacter and Francisella species. Also contemplated is the expressing of other polysaccharide antigens from any organism in the attenuated Salmonella strain. As these polysaccharides are often immunodominant and antibodies against them are frequently protective, this approach would permit delivery of these recombinant Salmonella strains by intranasal administration to protect against subsequent challenge. In addition, this system would be used in conjunction with any additional polysaccharide, protein, or glycoprotein expressed on Salmonella. Therefore, in accordance with the invention, combination vaccines able to express multiple epitopes from a single pathogen to produce a multivalent vaccine that could be more immunogenic when delivered intranasally are feasible. Additionally, it is likely that one could express epitopes from different pathogens, thereby minimizing the number of vaccinations required.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers. Such a formulation is administered in the manner in which snuff is taken, i.e. by rapid inhalation through the nasal passage from a container of the powder held close to the nares. Other methods are also available.

Formulations suitable for nasal administration may, for example, comprise from about as little as 0.1% (w/w) and as much as 100% (w/w) of the active ingredient, and may further comprise one or more of the additional ingredients described herein.

Typically, dosages of the compound of the invention which may be administered to an animal, preferably a human, range in amount from 1 μg to about 100 g per kilogram of body weight of the animal. While the precise dosage administered will vary depending upon any number of factors, including but not limited to, the type of animal and type of disease state being treated, the age of the animal, the route of administration and whether the compound is being used to treat an existing disease or to immunize against future infection. Preferably, the dosage of the compound will vary from about 1 mg to about 10 g per kilogram of body weight of the animal. More preferably, the dosage will vary from about 10 mg to about 1 g per kilogram of body weight of the animal.

The compound may be administered to an animal as frequently as several times daily, or it may be administered less frequently, such as once a day, once a week, once every two weeks, once a month, or even lees frequently, such as once every several months or even once a year or less, again dependent upon the nature of the treatment. The frequency of the dose will be readily apparent to the skilled artisan and will depend upon any number of factors, such as, but not limited to, the type and severity of the disease being treated, the type and age of the animal, etc.

Other methods which were used but not described herein are well known and within the competence of one of ordinary skill in the art of clinical medicine, microbiology, and molecular biology. One of skill in the art will appreciate that the superiority of the compositions and methods of the invention relative to the compositions and methods of the prior art are unrelated to the physiological accuracy of the theory explaining the superior results. Some examples of diseases or conditions which may be treated or prevented according to the methods of the invention are discussed herein. The invention should not be construed as being limited solely to these examples, as other diseases or conditions which are at present unknown, once known, may also be treatable using the methods of the invention.

While this invention has been disclosed with reference to specific embodiments, it is apparent that other embodiments and variations of this invention may be devised by others skilled in the art without departing from the true spirit and scope of the invention.

1. A method of treatment, said method comprising the intranasal administration of a vaccine having an active ingredient that is an attenuated Salmonella strain expressing a lipopolysaccharide O antigen from a Gram-negative pathogen.

2. The method of claim 1 where the pathogen is selected from the group consisting of Escherichia coli, Vibrio cholerae, Haemophilus influenzae, Moraxella catarrhalis, Neisseria meningitides, Bordetella pertussis, Yersinia, Burkholderia, Coxiella, Brucella, Shigella, Campylobacter, Francisella, and Pseudomonas aeruginosa species.

3. The method of claim 1 where the Salmonella strain is an attenuated strain of Salmonella typhimurium.

4. The method of claim 2 where the pathogen is Pseudomonas aeruginosa.

5. The method of claim 1 where the vaccine is in the form of a powder.

6. The method of claim 5 where the active ingredient has an average particle from about 0.2 to 500 micrometers.

7. The method of claim 1 where the formulation is administered by rapid inhalation through the nasal passage from a container of the powder held close to the nasal passageway.

8. The method of claim 5 where the active ingredient of the vaccine is present in a concentration of from 0.1% (w/w) to 100% (w/w).

9. The method of claim 5 where the dosage of the active ingredient is present in an amount of from 1 μg to about 100 g per kilogram of body weight.

10. The method of claim 9 where the dosage varies from about 1 mg to about 10 g per kilogram of body weight.

11. The method of claim 1 where the vaccine is administered several times daily.

12. A method of treatment, said method comprising the intranasal administration of a vaccine having an active ingredient that is an attenuated Salmonella strain expressing a lipopolysaccharide O antigen from Pseudomonas aeruginosa.

13. The method of claim 12 where the Salmonella strain is an attenuated strain of Salmonella typhimurium.

14. The method of claim 12 where the vaccine is in the form of a powder.

15. The method of claim 14 where the active ingredient has an average particle from about 0.2 to 500 micrometers.

16. The method of claim 12 where the formulation is administered by rapid inhalation through the nasal passage from a container of the powder held close to the nasal passageway.

17. The method of claim 16 where the active ingredient of the vaccine is present in a concentration of from 0.1% (w/w) to 100% (w/w).

18. The method of claim 12 where the dosage of the active ingredient is present in an amount of from 1 μg to about 100 g per kilogram of body weight.

19. The method of claim 18 where the dosage varies from about 1 mg to about 10 g per kilogram of body weight.

20. The method of claim 1 where the vaccine is administered several times daily.
